(19) United States
(12) Patent Application Publication
(10) Pub. No.: US 2018/0298442 A1
(43) Pub. Date: Oct. 18, 2018

(54) METHOD AND KIT FOR MAKING DIAGNOSIS OF MYOCARDIAL INFARCTION

(71) Applicant: Academia Sinica, Taipei (TW)

(72) Inventors: Patrick C.H. HSIEH, Taipei (TW); Chen-Yun CHEN, Taipei (TW)

(21) Appl. No.: 15/765,175

(22) PCT Filed: Sep. 30, 2016
(86) PCT No.: PCT/US16/54971

§ 371 Date: Mar. 30, 2018

Related U.S. Application Data
(60) Provisional application No. 62/234,672, filed on Sep. 30, 2015.

(51) Int. Cl. C12Q 1/6883

(52) U.S. Cl. CPC C12Q 1/6883; C12Q 2600/178

(57) ABSTRACT

Disclosed herein are a method and a kit for making a diagnosis as to whether a subject suffers from myocardial infarction (MI). The method and the kit can accurately and efficiently identify the MI patient through detecting the circulating let-7a and let-7f, both of which are highly expressed in healthy subject, and downregulated in MI patient. According to the present method and kit, the MI patient is capable of receiving a proper treatment in time.

### CROSS-REFERENCE TO RELATED APPLICATIONS

This application is a U.S. National Stage Filing under 35 U.S.C. 371 from International Patent Application Ser. No. PCT/US2016/054971, filed Sep. 30, 2016, and published on Apr. 6, 2017, which claims the priority of U.S. Ser. No. 62/234,672, filed Sep. 30, 2015, the disclosure of which are incorporated by reference herein in their entireties.

### BACKGROUND OF THE INVENTION

### 1. Field of the Invention

The present disclosure in general relates to the field of disease diagnosis. More particularly, the present disclosure relates to a method and a kit for making a diagnosis of myocardial infarction.

### 2. Description of Related Art

Myocardial infarction (MI, also known as heart attack) is a leading cause of morbidity and mortality worldwide. MI occurs when myocardial ischemia, a diminished blood supply to the heart, exceeds a critical threshold and overwhelms myocardial cellular repair mechanisms designed to maintain normal operating function and homeostasis. Ischemia at this critical threshold level for an extended period results in irreversible myocardial cell damage or death.

The symptoms of myocardial infarction include chest pain, radiation of chest pain into the jaw or teeth, shoulder, arm, and/or back, dyspnea or shortness of breath, epigastric discomfort with or without nausea and vomiting, diaphoresis or sweating, syncope, and impairment of cognitive function. Among those over 75 years old, about 5% have had an MI with little or no history of symptoms. MI may cause heart failure, irregular heartbeat, or cardiac arrest.

A number of markers are used in the diagnosis of MI, such as troponin I, troponin T, creatine kinase-MB (CK-MB), myoglobin, B-type natriuretic peptide (BNP), and C-reactive protein (CRP). However, the disadvantage of diagnostic procedures using these markers is that not all the patients can be successfully identified. Accordingly, there exists in the related art a need for an improved method for making a quick diagnosis of MI, which provides an accurate and sensitive means to identify the subject having or suspected of having MI, so that the subject in need thereof could receive a suitable therapeutic regimen or intervention in time.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The first aspect of the present invention pertains to a method of making a diagnosis from a blood sample of a subject as to whether the subject suffers from a MI. The method comprises:

(a) determining the amount of a target miRNA in the blood sample, wherein the target miRNA is let-7a or let-7f; and

(b) comparing the amount of the target miRNA in the blood sample with that of a control sample obtained from a healthy subject; wherein a lower amount of the target miRNA in the blood sample than that of the control sample indicates that the subject suffers from MI.

According to some embodiments of the present disclosure, the let-7a miRNA comprises a nucleic acid sequence at least 85% identical to SEQ ID NO: 1. According to other embodiments of the present disclosure, the let-7f miRNA comprises a nucleic acid sequence at least 85% identical to SEQ ID NO: 2.

According to the embodiment of the present disclosure, the blood sample evaluated by the present method can be a whole blood sample, a serum sample, or a plasma sample.

In one embodiment, the MI is ST elevation myocardial infarction (STEMI). In another embodiment, the MI is non-ST elevation myocardial infarction (NSTEMI).

According to certain embodiments of the present disclosure, the amount of the target miRNA can be determined by an assay selected from the group consisting of northern blotting, microarray, fluorescent assay, electrochemical assay, bioluminescent assay, bioluminescent protein reassembly, bioluminescence resonance energy transfer (BRET)-based assay, reverse transcription polymerase chain reaction (RT-PCR), fluorescence correlation spectroscopy, and surface-enhanced Raman spectroscopy. In one embodiment, the amount of the target miRNA is determined by RT-PCR with a polynucleotide (served as a primer) comprising a nucleic acid sequence at least 85% identical to SEQ ID NO: 3 or 4. In another embodiment, the amount of the target miRNA is determined by the microarray with a polynucleotide (served as a probe) comprising a nucleic acid sequence at least 85% identical to SEQ ID NO: 3 or 4.

The subject evaluated by present method is a mammal; preferably, the subject is a human.

The second aspect of the present invention is directed to a kit for detecting a target miRNA in a blood sample of a subject for determining whether the subject suffers from a MI. The kit comprises a polynucleotide and a hybridization buffer. According to the embodiments of the present disclosure, the polynucleotide comprises a nucleic acid sequence at least 85% identical to SEQ ID NO: 3 or 4, in which at least one nucleotide in the nucleic acid sequence is locked nucleic acid (LNA) nucleotide. In one preferred embodiment, the polynucleotide comprises a nucleic acid sequence at least 85% identical to SEQ ID NO: 3 or 4, in which at least seven nucleotides in the nucleic acid sequence are locked nucleic acid (LNA) nucleotides.

In one embodiment of the present disclosure, the kit further comprises a positive control sample obtained from a subject suffering from MI. In another embodiment of the present disclosure, the kit further comprises a negative control sample obtained from a healthy subject.

According to some embodiments of the present disclosure, the hybridization buffer of the present kit is selected from the group consisting of Tris-HCl/NaCl/MgCl₂ (TNM) buffer, phosphate-buffered saline (PBS) buffer, Tris-HCl buffer, saline sodium citrate (SSC) buffer, Hepes/EDTA/neocuproine (HEN) buffer, and Tris/EDTA/NaCl (TEN) buffer.

The third aspect of the present disclosure pertains to a device for detecting a target miRNA in a blood sample of a subject for determining whether the subject suffers from a myocardial infarction. The device comprises a nanopore or a biosensor that is configured to detect the target miRNA, wherein the target miRNA is let-7a or let-7f; and a processing unit that is configured to calculate the amount of the target miRNA in the blood sample.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIGS. 1A and 1B are histograms respectively depicting the expression of specified miRNAs in the let-7 family, according to small RNA-sequencing of pig hearts (FIG. 1A) and human hearts (FIG. 1B). TPM: transcript per million. In both results, let-7a and let-7f are the most abundant miRNAs among let-7 family.

FIGS. 2A and 2B are the data that depict the expression of let-7a in cardiomyocytes (CM) and non-cardiomyocytes (non-CM). FIG. 2A: let-7a expresses in sorted rat CM and non-CM. FIG. 2B: In situ staining indicates that let-7a is ubiquitously expressed in the heart tissue of mice.

FIGS. 3A and 3B are dot plots that depict the downregulation of let-7a and let-7f after MI in porcine model. FIG. 3A: Time course expression levels of let-7a and let-7f after MI using stem-loop qRCR method. FIG. 3B: The expression levels of let-7a (left panel) and let-7f (right panel) are respectively determined by TaqMan qPCR method. UR ratio indicates the expression level of let-7a or let-7f in the infarcted area to that in the remote zone. Using these two methods, the downregulation of let-7a and let-7f was found within 24 hrs post-MI.

FIGS. 4A and 4D are the data that depict the expression of let-7a and let-7b at specified time point post-MI surgery in the porcine model. FIG. 4A: The expression level of let-7a in the plasma of pigs before, one-day after or one-week after MI surgery. FIG. 4B: The expression level of let-7f in the plasma of pigs before, one-day after or one-week after MI surgery. Unpaired t-test (n=18, * indicates p<0.05). FIG. 4C: Change in the expression level of let-7a in the plasma of each pig after MI surgery. FIG. 4D: Change in the expression level of let-7f in the plasma of each pig after MI surgery. Paired t-test (n=18, * indicates p<0.05).

FIGS. 5A and 5B are dot plots that depict the downregulation of let-7a and let-7f in the plasma of patients with acute myocardial infarction (AMI). FIG. 5A: The expression of let-7a in 9 healthy subjects and 25 AMI patients. FIG. 5B: The expression of let-7f in 9 healthy subjects and 25 AMI patients. In these experiments, microRNA is extracted from 100 ul of plasma, and cel-mir-39 is used as a spike-in control.

In accordance with common practice, the various described features/elements are not drawn to scale but instead are drawn to best illustrate specific features/elements relevant to the present invention.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The terms “myocardial infarction” and “acute myocardial infarction” as used herein are interchangeable. The term relates to the irreversible necrosis of the myocardium as a result of prolonged ischemia. As it will be understood by those skilled in the art, the diagnosis is usually not intended to be correct for 100% of the subjects to be analyzed. The term, however, requires that the diagnosis will be valid for a statistically significant portion of the subjects to be examined. Whether a portion is statistically significant can be determined without further ado by the person skilled in the art using various well known statistic evaluation tools, e.g., determination of confidence intervals, p-value determination, Student's t-test, Mann-Whitney test, etc. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the diagnosis will be correct for at least 60%, at least 70%, at least 80%, or at least 90% of the subjects of a given cohort.

The term “miRNA” or “microRNA” is understood by the skilled artisan and relates to a short ribonucleic acid (RNA) molecule found in eukaryotic cells and in body fluids of metazoan organisms. It is to be understood that the present invention also encompasses pri-miRNAs, and the pre-miRNAs of the miRNAs of the present invention. Thus preferably, a miRNA-precursor consists of 25 to several thousand nucleotides, more preferably 40 to 130 nucleotides, even more preferably 50 to 120 nucleotides, or, most preferably 60 to 110 nucleotides. Preferably, a miRNA consists of 5 to 100 nucleotides, more preferably 10 to 50 nucleotides, even more preferably 12 to 40 nucleotides, or, most preferably 18 to 26 nucleotides. Preferably, the miRNAs of the present invention are miRNAs of human origin, i.e. they are encoded in the human genome. Also preferably, the term miRNA relates to the “guide” strand which eventually enters the RNA-induced silencing complex (RISC) as well as to the “passenger” strand complementary thereto. Moreover, any relation to a specific miRNA in this specification is, preferably, to be understood to include variants of the specific miRNA. Said variants may represent orthologs, paralogs or other homologs. Further, variants include polynucleotides comprising nucleic acid sequences which are at least 85% identical to the nucleic acid sequences of the specific miRNA sequences. The percent identity values are, preferably, calculated over the entire nucleic acid sequence region.

As used herein, the term “polynucleotide” or “nucleic acid sequence” refers to a single- or double-stranded polymer of RNA, deoxyribonucleic acid (DNA), or the combination thereof read from the 5′ to the 3′ end. In the present disclosure, the polynucleotide may comprise one or more modified nucleotide residues (e.g., locked nucleic acid (LNA) nucleotide) and may be used as primers or probes. A “primer” or “probe” refers to a polynucleotide (synthetic or occurring naturally) comprising a nucleic acid sequence that is complementary to a nucleic acid sequence present in a target molecule and can form a duplexed structure by hybridization with the target molecule. Typically, the term “primer” refers to a single-stranded polynucleotide complementary to a nucleic acid sequence sought to be copied and serves as a starting point for synthesis of a primer extension product, while the term “probe” refers to a single-stranded polynucleotide that can be hybridized with a complementary single-stranded target sequence to form a double-stranded molecule (hybrid).

As used herein, the term “hybridize” or “hybridization” refers to any process by which a strand of nucleic acid binds with a complementary strand through base pairing. In general, hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

The term “amount” as used herein encompasses the absolute amount of a miRNA referred to herein, the relative amount or concentration of a miRNA referred to herein, as well as any value or parameter which correlates thereto. Such values or parameters comprise intensity signal values from all specific physical or chemical properties obtained from the miRNA referred to herein by measurements, e.g., intensity signals obtained from specifically bound ligands. It is to be understood that values correlating to the aforementioned amounts or parameters can also be obtained by all standard mathematical operations.

As used herein, the term “locked nucleic acid” or “LNA” refers to bicyclic nucleic acid analogs contain one or more 2′-O, 4′-C methylene linkage(s), which effectively locks the furanose ring in a C3′-endo conformation. This methylene linkage restricts the flexibility of the ribofuranose ring and locks the structure into a rigid bicyclic formation. Because of its structural conformation, locked nucleic acids demonstrate a much greater affinity and specificity to their complementary nucleic acids than do natural DNA counterparts and increases thermal and chemical stability of a probe/target nucleic acid duplex. LNAs will hybridize to complementary nucleic acids even under adverse conditions, such as under low salt concentrations and in the presence of chaotropic agents. Locked nucleic acid nucleotides can be mixed with DNA or RNA bases in the oligonucleotide whenever desired; more specifically, locked nucleic acid nucleotides may be interspersed throughout an oligonucleotide sequence, placed consecutively or placed singularly in predetermined locations.

The term “sequence identity” as used herein refers to the sequence relationships between two or more nucleic acids or amino acid sequences when aligned for maximum correspondence over a specified comparison window. The percentage of “identity” is determined by comparing two optimally aligned sequences over the comparison window. For “optimal alignment” of the two sequences, it will be appreciated that the portion of the sequence in the comparison window may include gaps (e.g., deletions or additions) as compared to the reference sequence, which does not contain additions or deletions. After alignment, the number of matched positions (i.e., positions where the identical nucleic acid base or amino acid residue occurs in both sequences) is determined and then divided by the total number of positions in the comparison window. This result is then multiplied by 100 to calculate the percentage of sequence or amino acid identity. In some embodiments, two sequences have the same total number of nucleotides or amino acids. The aligned sequences can be analyzed by any method familiar with one skilled artisan, including GAP, BESTFIT, BLAST, FASTA, and TFASTA.

As the need for accurately and efficiently identifying whether a subject suffers from a MI, and accordingly, providing appropriate treatments in time, the objective of the present disclosure aims at providing a method and a kit for making a diagnosis of MI from a blood sample of a subject.

Thus, the first aspect of the present disclosure is directed to a method of making a diagnosis from a blood sample of a subject as to whether the subject suffers from MI. The method comprises,

(a) determining the amount of a target miRNA in the blood sample, wherein the target miRNA is let-7a or let-7f; and

(b) comparing the amount of the target miRNA in the blood sample with that of a control sample obtained from a healthy subject; wherein a lower amount of the target miRNA in the blood sample than that of the control sample indicates that the subject suffers from MI.

In the present method, a blood sample is first obtained from the subject having or suspected of having MI. According to the embodiments of the present disclosure, the blood sample can be a whole blood sample, a serum sample, or a plasma sample. In one specific example, the blood sample is the plasma sample.

In the step (a), a target miRNA in the blood sample is quantified by any suitable assay. According to examples of the present disclosure, total RNA is first extracted from the blood sample (e.g., the plasma sample) by any method that is known to the skilled artisan (such as using cell lysis buffer to release nucleic acids from the cells), or by use of commercial kits available for such purpose. Non-limiting examples of cell lysis buffer include, NP-40 lysis buffer, radiolmmunoprecipitation assay (RIPA) lysis buffer, sodium dodecyl sulfate (SDS) lysis buffer, and ammonium-chloride-potassium (ACK) lysis buffer. Examples of kits suitable for use in the present method include, but are not limited to, mirVana PARIS kit (Ambion), miRCURY RNA Isolation Kit (Exiqon), miRNeasy Serum/Plasma Kit (Qiagen), Total RNA Purification Kit (Norgen Biotek Corporation), and NucleoSpin miRNAs kit (Macherey-Nagel). According to one embodiment of the present disclosure, total RNA is extracted from the plasma sample by use of mirVana PARIS kit. The extracted RNA is then used for measuring or quantifying specified miRNA targets.

According to the embodiments of the present disclosure, suitable examples of the assay for determining the amount of the target miRNA include, but are not limited to, northern blotting, microarray, fluorescent assay, electrochemical assay, bioluminescent assay, bioluminescent protein reassembly, bioluminescence resonance energy transfer (BRET)-based assay, reverse transcription polymerase chain reaction (RT-PCR), fluorescence correlation spectroscopy, and surface-enhanced Raman spectroscopy. The amount of the target miRNA can be expressed as an absolute value or a relative value, depending on the quantification assay. In one embodiment of the present disclosure, the target miRNA is quantified by RT-PCR. In another embodiment of the present disclosure, the target miRNA is quantified by microarray or biochip.

According to some embodiments of the present disclosure, the target miRNA is let-7a, which comprises a nucleic acid sequence at least 85% identical to SEQ ID NO: 1; for example, let-7a may comprise a nucleic acid sequence that is 85%, 86%, 87%, 88%, 89%, 90%, 91%, 92%, 93%, 94%, 95%, 96%, 97%, 98%, 99%, or 100% identical to SEQ ID NO: 1. Preferably, the nucleic acid sequence comprised in let-7a is at least 90% identical to SEQ ID NO: 1. More preferably, the nucleic acid sequence comprised in let-7a is at least 95% identical to SEQ ID NO: 1. In one specific embodiment of the present disclosure, let-7a has the nucleic acid sequence of SEQ ID NO: 1.

In the embodiments of the present disclosure, the amount of the let-7a is determined by use of a polynucleotide acting as a probe, which comprises a nucleic acid sequence at least 85% identical to SEQ ID NO: 3. According to the embodiment, the nucleic acid sequence of SEQ ID NO: 3 is complementary to the nucleic acid sequence of SEQ ID NO: 1. Accordingly, the polynucleotide comprising the nucleic acid sequence of SEQ ID NO: 3 would exhibit binding affinity and specificity to let-7a that comprises the nucleic acid sequence of SEQ ID NO: 1.

According to embodiments of the present disclosure, the present polynucleotide may comprise one or more locked nucleic acid (LNA) nucleotides. Preferably, the present polynucleotide comprises at least 7 LNA nucleotides. In one example, the present polynucleotide comprises 7 LNA nucleotides, which are respectively located at bases 2, 5, 8, 11, 14, 17, and 20 of the nucleic acid sequence of SEQ ID NO: 3. In another example, the present polynucleotide comprises 7 LNA nucleotides, which are respectively located at bases 3, 6, 9, 12, 15, 18, and 21 of the nucleic acid sequence of SEQ ID NO: 3. In still another example, the present polynucleotide comprises 8 LNA nucleotides, which are respectively located at bases 1, 4, 7, 10, 13, 16, 19, and 22 of the nucleic acid sequence of SEQ ID NO: 3.

According to other embodiments of the present disclosure, the target miRNA is let-7f, which comprises a nucleic acid sequence at least 85% identical to the sequence of SEQ ID NO: 2; that means, the let-7f and the sequence of SEQ ID NO: 2 share a sequence identity that is 85%, 88%, 87%, 88%, 89%, 90%, 91%, 92%, 93%, 94%, 95%, 96%, 97%, 98%, 99%, or 100%. Preferably, the nucleic acid sequence comprised in let-7f is at least 90% identical to SEQ ID NO: 2. More preferably, the nucleic acid sequence comprised in let-7f is at least 95% identical to SEQ ID NO: 2. In one specific embodiment of the present disclosure, let-7f comprises a nucleic acid that is 100% identical to SEQ ID NO: 2.

In these embodiments, the amount of the let-7f is determined by a polynucleotide comprising a nucleic acid sequence at least 85% identical to SEQ ID NO: 4, which is designed to be complementary to the nucleic acid sequence of SEQ ID NO: 2. Thus, the polynucleotide comprising the nucleic acid sequence of SEQ ID NO: 4 is capable of specifically binding to let-7f that comprises the nucleic acid sequence of SEQ ID NO: 2. Preferably, the nucleic acid sequence of the present polynucleotide comprises at least 7 LNA nucleotides. In one example, the nucleic acid sequence of the present polynucleotide comprises 7 LNA nucleotides, which are respectively located at bases 2, 5, 8, 11, 14, 17, and 20 of the nucleic acid sequence of SEQ ID NO: 4. In another example, the nucleic acid sequence of the present polynucleotide comprises 7 LNA nucleotides, which are respectively located at bases 3, 6, 9, 12, 15, 18, and 21 of the nucleic acid sequence of SEQ ID NO: 4. In still another example, the nucleic acid sequence of the present polynucleotide comprises 8 LNA nucleotides, which are respectively located at bases 1, 4, 7, 10, 13, 16, 19, and 22 of the nucleic acid sequence of SEQ ID NO: 4.

Based on the characterization of LNA nucleotides, the targeting miRNA (e.g., let-7a or let-7f) can be hybridized with the polynucleotide comprising LNA nucleotide(s) under adverse conditions, such as low salt concentration and/or high temperature. According to one embodiment of the present disclosure, the targeting miRNA is hybridized with the polynucleotide comprising LNA nucleotides at 50-60° C.

According to some examples of the present disclosure, the present polynucleotide may be immobilized on a solid support, such as magnetic bead, glass, or silicon, prior to binding with the target miRNA (i.e., let-7a or let-7f).

The complementary base pairing between polynucleotide and target miRNA forms a “double strand” structure. The RNA-DNA hybrids can be detected by any method familiar with a skilled artisan. Examples of methods for the detection of RNA-DNA hybrids include, but are not limited to, polyamide method (by use of a small chemical compound conjugated with a fluorescent dye or a specific substrate, in which the small chemical compound can be designed to bind with sequence specificity in the minor groove of RNA-DNA hybrids), triplex method (by use of a triple helix-forming oligonucleotide conjugated with a fluorescent dye or a radiation molecule, in which the triple helix-forming oligonucleotide is designed to bind to the polypurine/polypyrimidine tracts in the major groove of dsDNA), and protein method (by use of a sequence-specific DNA binding protein conjugated with a fluorescent dye or a specific substrate).

According to one working example, a polynucleotide comprising the sequence (i.e., SEQ ID NO: 3) complementary to the nucleic acid sequence of let-7a (i.e., SEQ ID NO: 1), or the sequence (i.e., SEQ ID NO: 4) complementary to the nucleic acid sequence of let-7f (i.e., SEQ ID NO: 2) is first immobilized on a solid support. Then, the plasma sample obtained from the peripheral bloods of MI patient was hybridized with the polynucleotide at 50-60° C. so as to form a RNA-DNA hybrid. The RNA-DNA hybrid could be detected by adding a dye or fluorescence that incorporated into the RNA-DNA hybrid structure. According to the signal emitted by the incorporating dye or fluorescence, the expression level of let-7a or let-7b could be quantifiably analyzed.

Next, the amount of the target miRNA (i.e., let-7a or let-7f) in the blood sample is compared with that of a control sample obtained from a healthy subject as described in step (b). According to the embodiments of the present disclosure, a lower amount of the target miRNA in the blood sample than that of the control sample indicates that the subject suffers from MI.

Optionally, a synthetic RNA can be used as a spike-in (internal) control to quantify the amount of target miRNA. The normal distribution of miRNA amount will be established from healthy subjects. Once the amount of miRNA from a subject is determined, the result can be referred to normal distribution table.

According to one embodiment, MI evaluated by the present method is ST elevation myocardial infarction (STEMI). According to another embodiment, MI evaluated by the present method is non-ST elevation myocardial infarction (NSTEMI).

According to certain embodiments of the present disclosure, the subject evaluated by the present method is a mammal, including a human, a chimp, a monkey, a dog, a pig, a rat, and a mouse. In one specific example, the subject evaluated by the present method is a human.

As would be appreciated, the target miRNA can be the combination of let-7a and let-7f. In such case, the total amount of let-7a and let-7f in the blood sample are compared with that of the control sample obtained from the healthy subject. When the total amount of let-7a miRNA and let-7a miRNA in the blood sample are lower than that of the control sample, then the subject would be diagnosed as a patient suffering from MI (i.e., MI patient).

The second aspect of the present disclosure pertains to a kit for detecting a target miRNA in a blood sample of a subject for determining whether the subject suffers from a MI. The kit comprises a polynucleotide and a hybridization buffer.

According to some embodiments of the present disclosure, the polynucleotide comprises a nucleic acid sequence at least 85% identical to SEQ ID NO: 3. To enhance the sensitivity and specificity, at least one nucleotide in the nucleic acid sequence of the present polynucleotide is modified as LNA nucleotide; for example, the nucleic acid sequence of the present polynucleotide may comprises 1, 2, 3, 4, 5, 6, 7, 8, 9, 10, 11, 12, 13, 14, 15, 16, 17, 18, 19, 20, 21, or 22 LNA nucleotide(s). Preferably, the nucleic acid sequence of the present polynucleotide comprises at least 7 LNA nucleotides. In one example, the nucleic acid sequence of the present polynucleotide comprises 7 LNA nucleotides, which are respectively located at bases 2, 5, 8, 11, 14, 17, and 20 of the nucleic acid sequence of SEQ ID NO: 3. In another example, the nucleic acid sequence of the present polynucleotide comprises 7 LNA nucleotides, which are respectively located at bases 3, 6, 9, 12, 15, 18, and 21 of the nucleic acid sequence of SEQ ID NO: 3. In still another example, the nucleic acid sequence of the present robe comprises 8 LNA nucleotides, which are respectively located at bases 1, 4, 7, 10, 13, 16, 19, and 22 of the nucleic acid sequence of SEQ ID NO: 3.

According to some embodiments of the present disclosure, the polynucleotide comprises the nucleic acid sequence at least 85% identical to SEQ ID NO: 4, in which at least one nucleotide in the nucleic acid sequence is the LNA nucleotide; for example, the nucleic acid sequence of the present polynucleotide may comprises 1, 2, 3, 4, 5, 6, 7, 8, 9, 10, 11, 12, 13, 14, 15, 16, 17, 18, 19, 20, 21, or 22 LNA nucleotide(s). Preferably, the nucleic acid sequence of the present polynucleotide comprises at least 7 LNA nucleotides. In one example, the nucleic acid sequence of the present polynucleotide comprises 7 LNA nucleotides, which are respectively located at bases 2, 5, 8, 11, 14, 17, and 20 of the nucleic acid sequence of SEQ ID NO: 4. In another example, the nucleic acid sequence of the present polynucleotide comprises 7 LNA nucleotides, which are respectively located at bases 3, 6, 9, 12, 15, 18, and 21 of the nucleic acid sequence of SEQ ID NO: 4. In still another example, the nucleic acid sequence of the present polynucleotide comprises 8 LNA nucleotides, which are respectively located at bases 1, 4, 7, 10, 13, 16, 19, and 22 of the nucleic acid sequence of SEQ ID NO: 4.

In one embodiment, the polynucleotide has a hairpin-loop structure. In another embodiment, the polynucleotide has a stem-loop structure. In still another embodiment, the polynucleotide has a double-loop structure.

According to some embodiments of the present disclosure, the kit further comprises a positive control sample obtained from a subject suffering from MI or from a synthetic RNA. According to other embodiments of the present disclosure, the kit further comprises a negative control sample obtained from a healthy subject or from a synthetic RNA.

According to the embodiments of the present disclosure, the hybridization buffer is selected from the group consisting of Tris-HCl/NaCl/MgCl₂ (TNM) buffer, phosphate-buffered saline (PBS) buffer, Tris-HCl buffer, saline sodium citrate (SSC) buffer, Hepes/EDTA/neocuproine (HEN) buffer, and Tris/EDTA/NaCl (TEN) buffer.

The third aspect of the present disclosure pertains to a device for detecting a target miRNA in a blood sample of a subject for determining whether the subject suffers from a myocardial infarction. The device comprises a nanopore or a biosensor that is configured to detect the target miRNA, wherein the target miRNA is let-7a or let-7f; and a processing unit that is configured to calculate the amount of the target miRNA in the blood sample.

According to some embodiments of the present disclosure, the let-7a miRNA comprises a nucleic acid sequence at least 85% identical to SEQ ID NO: 1. According to other embodiments of the present disclosure, the let-7f miRNA comprises a nucleic acid sequence at least 85% identical to SEQ ID NO: 2.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

Materials and Methods

Porcine Model of Myocardial Infarction

Surgical anesthesia was introduced in about 22 Kg Lanyu minipigs by use of Zoletil (12.5 mg/kg; Virbac, France), Rompun (0.2 ml/kg; Bayer Healthcare, Germany), and atropine (0.05 mg/kg; TBC, Taiwan) before intubation. They were attached to a respirator for intermittent positive pressure ventilation with a mixture of oxygen, air, and Isoflurane (1.5 to 2%; Baxter Healthcare, Guayama, PR). For the sustained administration of saline or anesthetic, a venous indwelling catheter was maintained in an ear vein during surgery. After surgery, an antibiotic (Ampolin, YSP) to prevent infection and an analgesic to alleviate pain (Keto, YSP) were given. MI surgery was performed by permanent occlusion at the midleft anterior descending artery.

miRNA Extraction

The plasma sample was isolated from the peripheral blood through centrifugation. Then, total RNA was extracted from the plasma sample by use of mirVana PARIS kit (Ambion) following the manufacturer's instructions. Synthetic microRNA named cel-mir-39 was added into the samples as an internal control for normalization of technical variations.

Stem-Loop Quantitative Polymerase Chain Reaction (qPCR)

Fifty nano-grams of total RNA were subjected to reverse transcription using Taqman microRNA reverse transcription kit (Applied Biosystems) with synthesized oligonucleotide of SEQ ID NO: 5, which form stem loop structure and antisense of microRNA as RT primer. Quantitative PCR was performed using a forward primer of SEQ ID NO: 6 or 7, and a universal reward primer of SEQ ID NO: 8 in OmicsGreen qPCR Master Mix (Omics Bio) with an ABI 7500 real-time PCR system (Applied Biosystems).

Taqman Quantitative Polymerase Chain Reaction

The Taqman microRNA reverse transcription kit (Applied Biosystems) and the Taqman Universal PCR master mix (Applied Biosystems) were used. Stem loop RT primers for individual microRNA were provided with TaqMan microRNA probes. Stem loop RT was performed using TaqMan microRNA reverse transcription kit (Assay ID 000377 for the detection of let-7a and Assay ID 000382 for the detection of let-7f). The quantitative PCR procedures were carried out following manufacturer's instructions provided with the Taqman microRNA assays (Applied Biosystems).

### Example 1 Expression of let-7a or let-7f in Healthy Subject

In this example, the expressions of let-7 family miRNAs (including let-7a, let-7b, let-7c, let-7d, let-7e, let-7f, let-7g and let-7i miRNAs) in porcine, human and rodents were respectively measured in accordance to procedures described in the “Materials and Methods” section.

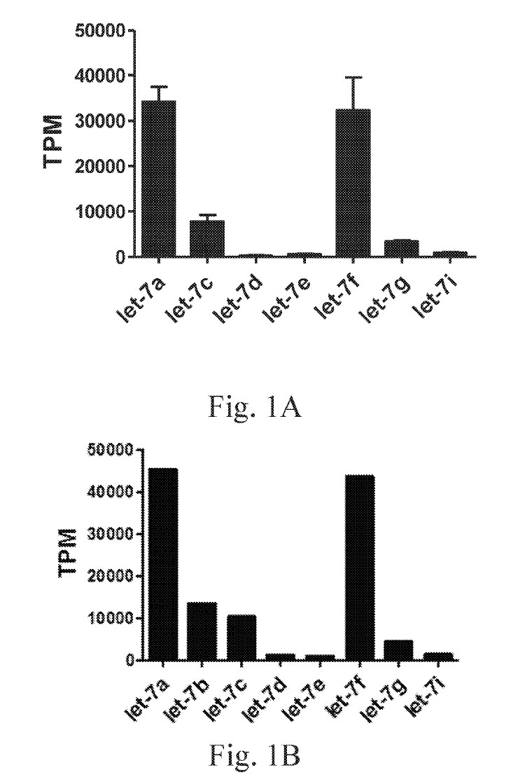

The expressions of let-7 miRNAs in the heart of porcine were first analyzed by small RNA-sequencing assay. As the data of FIG. 1A indicated, the expressed levels of let-7a and let-7f were the highest among the let-7 gene family. The expressions of let-7 miRNA derived from the heart of human exhibited the similar result with that observed in porcine (FIG. 1 B).

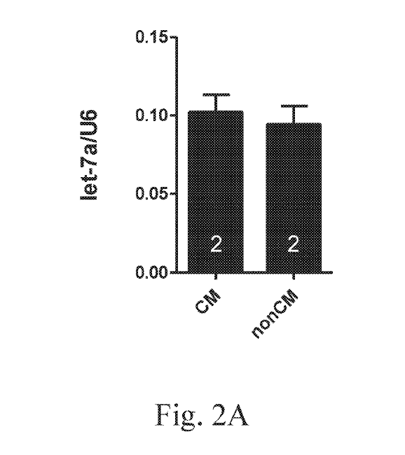

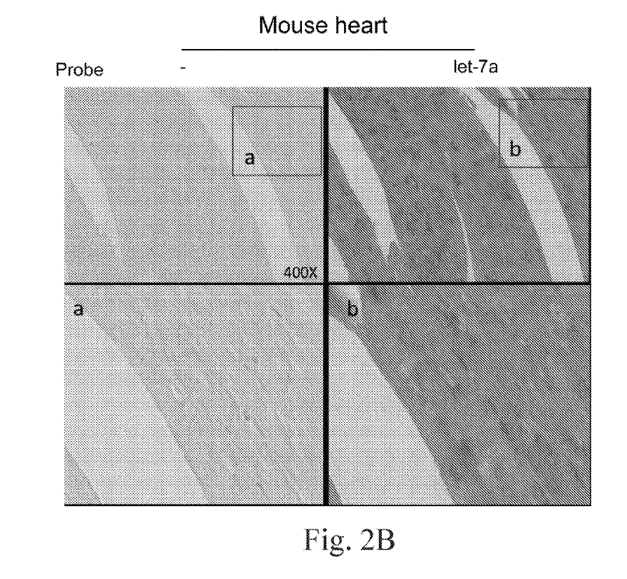

Based on the expression profiles of let-7 miRNAs detected in porcine and human, the expression of let-7a was further examined in rodents by tetramethylrhodamine methyl ester (TMRM) staining followed by flow cytometry analysis, and in situ staining. The results were respectively depicted in FIGS. 2A and 2B. The data of TMRM staining indicated the expression of let-7a could be detected in both rat cardiomyocyte (CM) and rat cardiac fibroblast (non-CM) (FIG. 2A). The result of in situ staining further confirmed the ubiquitous expression of let-7a in heart tissue of mice (FIG. 2B).

### Example 2 Expression of let-7a or let-7f in Subject Suffering from myocardial Infarction (MI)

The data of example 1 illustrated that both let-7a and let-7f were highly expressed in the heart tissue of healthy animals. In this example, the effects of MI on the expression of both let-7a and let-7f were investigated using a porcine MI model (example 2.1) and human subjects (example 2.2), respectively.

### 2.1 Porcine Model of myocardial Infarction (MI)

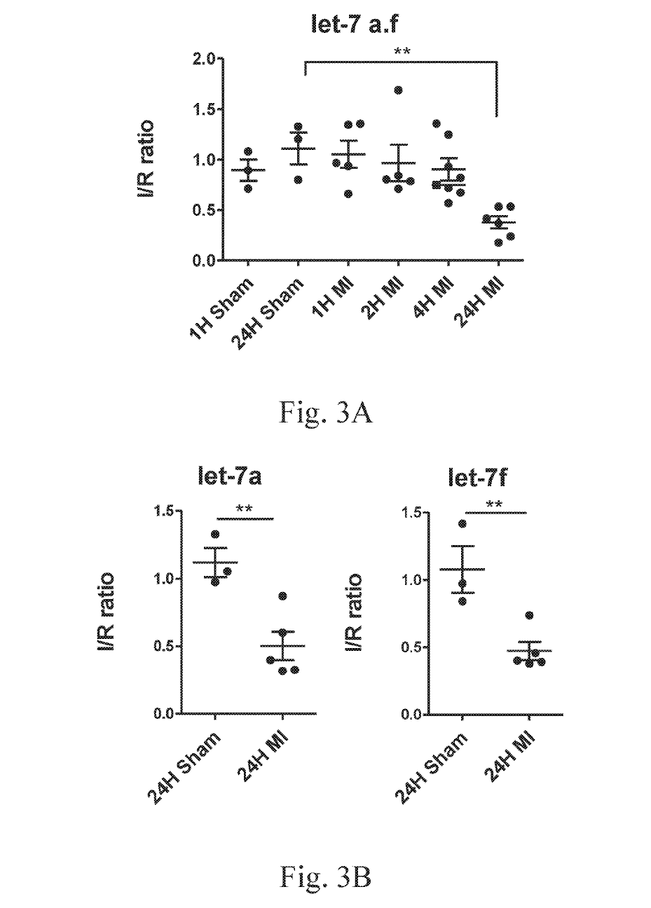

In example 2.1, a porcine MI model was created in accordance with the procedures described in “materials and Methods” section, and the levels of let-7a and let-7f in the infarcted area and a remote area were measured, respectively. Results were depicted in FIGS. 3A and 3B.

FIG. 3A was the time course expressions of both let-7a and let-7f after MI surgery. The data of stem-loop qPCR indicated that compared with the control group (Sham group), the combined level of let-7a and let-7f in the MI pigs (i.e., the MI group) decreased gradually within the first 24 hrs post-MI, and the decrease was statistically significant as compared to that of the shame group. UR ratio indicated the ratio of normalized expression level between ischemic region and remote region.

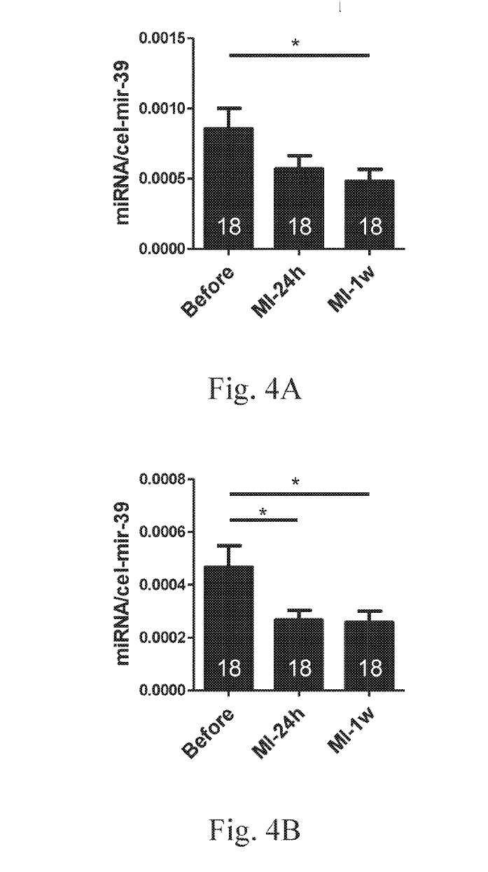

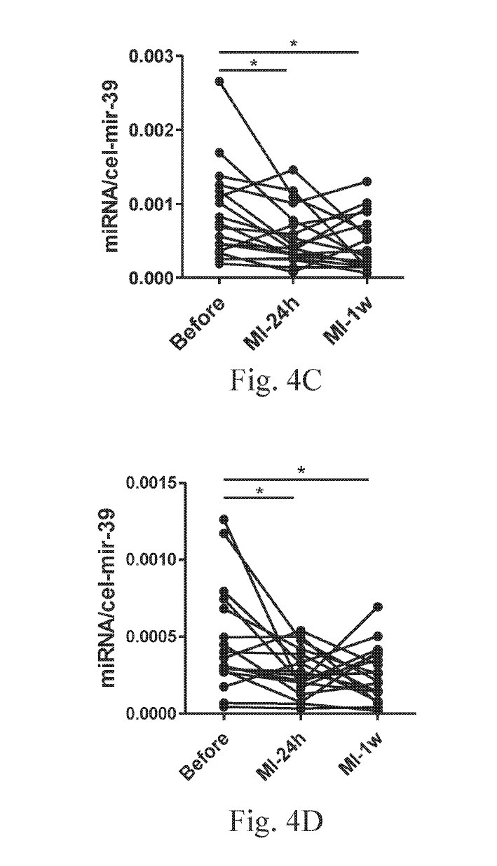

The respective levels of let-7a and let-7f were confirmed by TaqMan qPCR method, in which the expression of let-7a (FIG. 3B, left panel) and let-7f (FIG. 3B, right panel) in MI group were respectively significantly lower than that of the Sham group. The data of FIGS. 4A-4D further revealed that the downregulation of plasma let-7a (FIGS. 4A and 4C) and let-7f (FIGS. 4B and 4D) in the MI pigs lasted for at least one week post-MI.

### 2.2 Human Subject Having myocardial Infarction

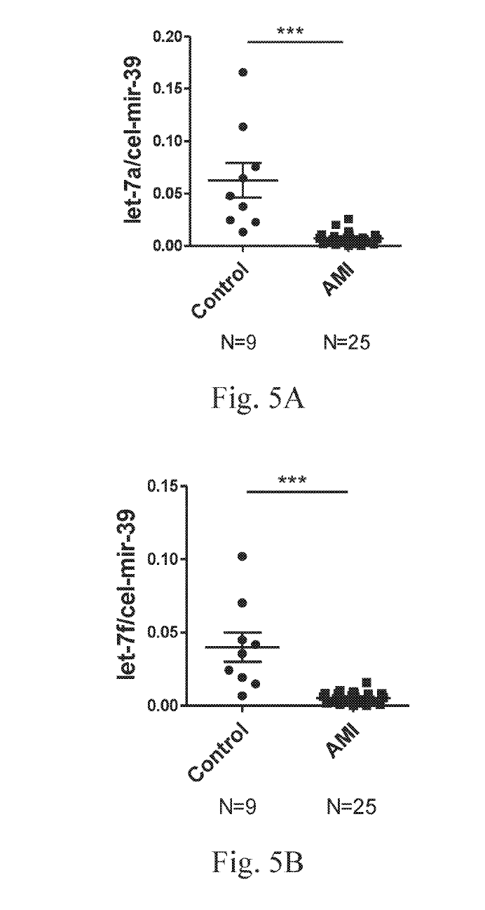

In this example, the plasma samples were respectively obtained from 9 healthy subjects and 25 MI patients with informed written consent, and the levels of left-7a and left-7f were respectively measured. Results were depicted in FIGS. 5A and 5B.

As depicted in FIG. 5, compared with healthy subjects (i.e., Control group), both let-7a (FIG. 5A) and let-7f (FIG. 5B) were significantly downregulated in the MI patients (i.e., AMI group).

Taken together, data in FIGS. 3-5 indicate that both let-7a and let-7f are contiguously expressed in the tissue and/or blood (e.g., heart tissue and plasma) of a healthy subject (e.g., porcine, rat, mouse, and human being), in which relatively higher levels of let-7a and let-7f could be measured; however, the expressions of both let-7a and let-7f in the heart tissue and/or blood were suppressed or downregulated once the subject has had MI.

In conclusion, the present disclosure provides a method and a kit for identifying the MI patients. Compared with other detection methods, which mostly analyze the miRNA extracted from heart tissue, the present method and kit is useful in directly quantifying the expression level of miRNA (i.e., let-7a and/or let-7f) from peripheral blood. According to the results, a practitioner can accurately and efficiently make a diagnosis as to whether the subject suffers from MI; and hence, providing a proper and prompt treatment to the MI patient.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1-10. (canceled)

11. A kit for detecting a target miRNA in a blood sample of a subject for determining whether the subject suffers from a myocardial infarction, comprising,
a polynucleotide comprising a nucleic acid sequence at least 85% identical to SEQ ID NO: 3 or 4, wherein at least one nucleotide in the nucleic acid sequence is a locked nucleic acid (LNA) nucleotide; and
a hybridization buffer.

12. The kit of claim 11, wherein at least seven nucleotides in the nucleic acid sequence of SEQ ID NO: 3 or 4 are locked nucleic acid (LNA) nucleotides.

13. The kit of claim 11, further comprising a positive control sample obtained from a subject suffering from myocardial infarction or from a synthetic RNA.

14. The kit of claim 11, further comprising a negative control sample obtained from a healthy subject or from a synthetic RNA.

15. The kit of claim 11, wherein the hybridization buffer is selected from the group consisting of Tris-HCl/NaCl/MgCl₂ (TNM) buffer, phosphate-buffered saline (PBS) buffer, Tris-HCl buffer, saline sodium citrate (SSC) buffer, Hepes/EDTA/neocuproine (HEN) buffer, and Tris/EDTA/NaCl (TEN) buffer.

16-18. (canceled)


SEQUENCE LISTING (8 sequences)

SEQ ID NO: 1 - RNA, 22 nt, Artificial Sequence
  let-7a
ugagguaguagguuguauaguu

SEQ ID NO: 2 - RNA, 22 nt, Artificial Sequence
  let-7f
ugagguaguagauuguauaguu

SEQ ID NO: 3 - DNA, 22 bp, Artificial Sequence
  anti-let-7a
aactatacaacctactacctca

SEQ ID NO: 4 - DNA, 22 bp, Artificial Sequence
  anti-let-7f
aactatacaatctactacctca

SEQ ID NO: 5 - DNA, 44 bp, Artificial Sequence
  RT-primer
ctcaactggtgtcgtggagtcggcaattcagttgagaactatac

SEQ ID NO: 6 - DNA, 30 bp, Artificial Sequence
  Forward Primer-1
acactccagctgggtgaggtagtagattgt

SEQ ID NO: 7 - DNA, 30 bp, Artificial Sequence
  Forward Primer-2
acactccagctgggtgaggtagtaggttgt

SEQ ID NO: 8 - DNA, 24 bp, Artificial Sequence
  Universal reward primer
ctggtgtcgtggagtcggcaattc